(19) United States
(12) Patent Application Publication
(10) Pub. No.: US 2004/0127986 A1
(43) Pub. Date: Jul. 1, 2004

(54) Ionic electroactive graft copolymer with a fluorine-containing backbone and a carbazole-containing side chain, blend thereof and actuator

(75) Inventors: Jen-Luan Chen, Hsinchu (TW); Wen-Liang Liu, Hsinchu (TW); Long-Cheng Cheng, Hsinchu (TW)

Correspondence Address: BACON & THOMAS, PLLC, 625 SLATERS LANE, FOURTH FLOOR, ALEXANDRIA, VA, 22314

(73) Assignee: Industrial Technology Research Institute, Hsinchu (TW)

(21) Appl. No.: 10/373,720

(22) Filed: Feb. 27, 2003

(30) Foreign Application Priority Data
Dec. 25, 2002 (TW) 91137375

(51) Int. Cl. A61F 2/02; C08F 26/12

(52) U.S. Cl. 623/11.11; 526/263

(57) ABSTRACT

An ionic electroactive graft copolymer with a fluorine-containing backbone and a carbazole-containing side chain having the following repeating unit is disclosed:

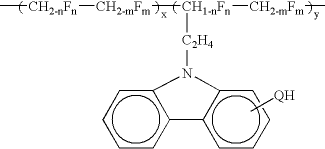

wherein n=0 or 1; m=0-2; x: y=3:1 to 35:1; and Q is anionic group, such as QH is —SO₃H. This copolymer or a blend thereof has a high conductivity, and is suitable for making an actuator and artificial muscles.

### FIELD OF THE INVENTION

The present invention is related to an ionic electroactive graft copolymer, and in particular to an ionic electroactive graft copolymer with a fluorine-containing backbone and a carbazole-containing side chain, which is suitable for making an actuator and artificial muscles.

### BACKGROUND OF THE INVENTION

Ionic electroactive polymer (abbreviated as ionic EAP) has advantages such as light weight, good elasticity, large deformation without fracture and good vibration damping. An electroactive polymer composite (EAPC) made of the ionic EAP and metal electrodes can be actuated by a low voltage, and has been utilized in the fabrications of various actuators such as a gripper, a microminiature pump, a microminiature fans, an electro-optical switch, and a smart valve; and artificial muscles capable of undergoing deformations resembling the behave of biological muscles. At present the most popular ionic EAP material is Nafion®; however this material still suffers certain disadvantages such as a low mechanical energy density, the actuation mechanisms and control parameters of actuation being not clear, relatively lower response time compared to the biological muscles, occurrence of residual deformation after driven by a DC voltage, and expensive. Further, an actuator made from Nafion® has a relatively higher liquid loss at room temperature, which can not be effectively overcome even with a containment made of silicone. In the fabrication of an EAPC a sand blasting treatment is required to enhance the deposition of metal electrodes on the surface of the perfluoride compoud Nafion®, which is not cost effective. U.S. Pat. No. 6,109,852 discloses a soft actuator and artificial muscles made from Nafion®.

### SUMMARY OF THE INVENTION

A primary objective of the present invention is to provide new ionic EAP materials. The new ionic EAP materials synthesized according to the present invention comprise a graft copolymer with a fluorine-containing backbone and a carbazole-containing side chain, and a polymer blend thereof. The new ionic EAP materials of the present invention have a conductivity, water uptake and mechanical properties comparable to Nafion®, but a faster response time and a greater force upon actuation at room temperature. Further, the new ionic EAP materials of the present invention are more suitable for making actuators and artificial muscles, because they are relatively easy to be processed and cheaper in price.

An ionic electroactive graft copolymer synthesized according to the present invention comprises the following repeating unit:

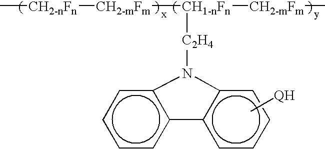

wherein n=0 or 1; m=0-2; x: y=3:1 to 35:1; and Q is an ionic group.

Preferably, the ionic electroactive graft copolymer of the present invention has a number average molecular weight of 80,000-350,000, or a weight average molecular weight of 144,000-700,000.

Preferably, n=0 and m=2.

Preferably, Q is —SO₃⁻.

The present invention also provides a polymer blend comprises the ionic electroactive graft copolymer of the present invention and a resin, wherein said resin is selected from the group consisting of polyvinylidene fluoride (PVdF), polysulfone, polyether ether ketone, polyethylene oxide, PVdF/polyhexafluorine propylene copolymer, PVdF/poly(chlorinetrifluorine ethylene) copolymer, and sulfonated PVdF-g-polystyrene, wherein the polymer blend comprises 1-70 wt % of said resin.

Preferably, said resin of said polymer blend is polyvinylidene fluoride.

The present invention further provides an actuator comprising a membrane and metal electrodes formed on two sides of said membrane, wherein said membrane comprises the ionic electroactive graft copolymer or the polymer blend of the present invention.

Preferably, said metal electrodes are platinum.

### DETAILED DESCRIPTION OF THE PREFERRED EMBODIMENTS

The present invention can be further understood from the following preferred embodiments, which are merely for illustrative not limitation of the scope of the present invention.

### EXAMPLE 1

Synthesis of Ionic Electroactive Polymeric Membrane

To 150 ml flask 4.8 g N-vinylcarbazole monomer (TCI Co., melting point 65° C., purity >98%), 8.0 g polyvinylidene fluoride (PVdF) having a number average molecular weight of 140,000 (Polysciences Co.) and 30 ml tetrahydrofuran (THF) (Pharmco Products Inc., Reagent Grade ACS) were added and well stirred by a magnetic stirrer at room temperature. The mixture was irradiated by Co-60 with a dosage of 20 kGy at room temperature to undergo a grafting reaction. The resulting crude product of PVdF-g-(N-ethylene carbazole was subjected to a Soxhlet extraction treatment with 20 ml trichlorinemethane, so that the remaining unreacted monomer and the styrene homopolymer were removed. The resulting purified product was dried in an oven at 60° C. and under atmospheric pressure for 6 hours to obtain 12.2 g of a light brown product, PVdF-g-(N-ethylene carbazole). The graft ratio by weight is 52.5%, which is defined as follows: [(weight of the resulting graft polymer)—(weight of PVdF)]/(weight of PVdF).

To a 500 ml flask 6.1 g of the above-prepared PVdF-g-(N-ethylene carbazole, 11.5 g of the above-mentioned PVdF, 15 mg of a fluorine-containing surfactant FC430 available from 3M, and 350 ml of 1-methyl-2-pyrrolidone) (TEDIA Co., Inc., HPLC grade) were added, and well stirred by a magnetic stirrer at 70° C. until a homogenous solution was formed. 15 ml of the resulting solution was cast on a glass substrate and heated by a heating plate at 120° C. to form a polymer blend membrane having a thickness of 200 μm and a diameter of 6 cm. The membrane was then sulfonated with chlorosulfonic acid (WAKO Co., purity 97%) at 25° C. for 8 hours. The sulfonated membrane was washed with 30 ml THF once and deionized water several times until the effluent was neutral. The membrane after swelling had a thickness of 230 μm. The conductivity of the membrane was measured according to the two-probe method with an AC Impedance Spectrometer with a combination of Solartron 1287 and 1260, and the result is 0.1379 S/cm.

The Making of Electrodes

Platinum electrodes were formed by the impregnation-reduction deposition method. The membrane prepared above was impregnated in 100 ml of 1M NaOH aqueous solution at room temperature for 24 hours, so that it was ion exchanged into a sodium salt form. The ion exchanged membrane was removed from the solution, and was impregnated in sequence in 45 ml of (Pt(NH₃)₄)Cl₂ aqueous solution (4 mgPt/ml) and 1 ml ammonia water (5 vol %) overnight. The impregnated membrane was removed, washed with deionized water to remove the residual solution from its surfaces, and then placed in a reduction tank having therein 180 ml deionized water. To the reduction tank 2 ml of sodium boron hydride solution (5 wt %) was added while stirring, and the temperature was controlled at 40° C. The same amount of sodium boron hydride solution was added at an interval of 30 minutes for a total of seven times. The reaction temperature was raised to 60° C. 30 minutes after the last addition, and a further 20 ml of sodium boron hydride solution (5 wt %) was added. Platinum electrodes were formed by reduction after maintaining the reaction temperature at 60° C. for 1.5 hours. The deposited membrane was taken from the reduction tank and soaked in 100 ml of 0.1 N HCl aqueous solution for one hour, and in 1 M NaOH aqueous solution for 24 hours to complete the making of the electroactive polymer composite. An artificial muscle element of 30 mm×3 mm (L×W) cutting from the resulting electroactive polymer composite was tested with a load cell (Transducer Technology Ltd., sn 1130487) to measure its tip force, and the measured tip force is 0.367 g with a displacement of 25 mm.

Control

Nafion® 117 membrane (E. I. duPont de Nemours & Co., Inc.) having a diameter of 6 cm was sand blasted for 10 minutes (2.5 Kg/cm²) to enhance the subsequent deposition of metal electrodes. The sand blasted membrane was washed with 150 ml deionized water three times, and heated in 100 ml 3 vol % H₂O₂ aqueous solution at 70° C. for one hour. The membrane was removed from the H₂O₂ aqueous solution, and soaked in 150 ml deionized water at room temperature three times with each time of one hour. The membrane was then soaked in 60 ml of 1N H₂SO₄ aqueous solution for 40 minutes, and in 150 ml deionized water at room temperature three times with each time of one hour. The resultant swelling membrane has a thickness of 200 μm, a conductivity measured at 25° C. of 0.0819 S/cm.

The impregnation-reduction deposition method described in Example 1 was repeated to form platinum electrodes on the swelling Nafion® 117 membrane. An artificial muscle element was prepared from the platinum deposited Nafion® 117 membrane composite and tested similarly as in Example 1. The measured tip force is 0.120 g with a displacement of 20 mm.

The following table lists the results of example 1 and contorl:

membrane

| | | PVdF-g- |
|---|---|---|
| | | (N-ethylene |
| Properties | Nafion ®/Pt | carbazole/Pt |
| Conductivity (S/cm)^{a} | 0.0819 | 0.1379 |
| Swelling ratio (%)^{a} | 20 | 25 |
| Actuation voltage (volt, | 5 | 5 |
| 0.5 Hz) | | |
| Displacement (mm) | 20^{b} | 25^{c} |
| Tip force (g) | 0.120^{b} | 0.367^{c} |

^{a}measured at 25° C. in deionized water, prior to the deposition of metal elecrtrodes
^{b}dimension of the artificial muscle element: 30 mm × 3 mm × 0.2 mm
^{c}dimension of the artificial muscle element: 30 mm × 3 mm × 0.23 mm

1. An ionic electroactive graft copolymer comprising the following repeating unit: 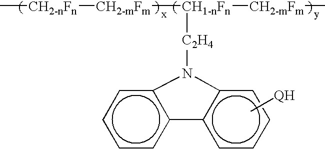
wherein n=0 or 1; m=0-2; x: y=3:1 to 35:1; and Q is an ionic group.

2. The ionic electroactive graft copolymer of claim 1, which has a number average molecular weight of 80,000-350,000, or a weight average molecular weight of 144,000-700,000.

3. The ionic electroactive graft copolymer of claim 1, wherein n=0 and m=2.

4. The ionic electroactive graft copolymer of claim 1, wherein Q is —SO₃⁻.

5. A polymer blend comprising the ionic electroactive graft copolymer defined in claim 1 and a resin, wherein said resin is selected from the group consisting of polyvinylidene fluoride (PVdF), polysulfone, polyether ether ketone, polyethylene oxide, PVdF/polyhexafluorine propylene copolymer, PVdF/poly(chlorinetrifluorine ethylene) copolymer, and sulfonated PVdF-g-polystyrene, wherein the polymer blend comprises 1-70 wt % of said resin.

6. The polymer blend of claim 5, wherein said resin is polyvinylidene fluoride.

7. The polymer blend of claim 5, wherein said ionic electroactive graft copolymer has a number average molecular weight of 80,000-350,000, or a weight average molecular weight of 144,000-700,000.

8. The polymer blend of claim 5, wherein n=0 and m=2.

9. The polymer blend of claim 5, wherein Q is —SO₃⁻.

10. An actuator comprising a membrane and metal electrodes formed on two sides of said membrane, wherein said membrane comprises the ionic electroactive graft copolymer defined in claim 1.

11. The actuator of claim 10, wherein said metal electrodes are platinum.

12. The actuator of claim 10, wherein said ionic electroactive graft copolymer has a number average molecular weight of 80,000-350,000, or a weight average molecular weight of 144,000-700,000.

13. The actuator of claim 10, wherein n=0 and m=2.

14. The actuator of claim 10, wherein Q is —SO₃^{—}.

15. An actuator comprising a membrane and metal electrodes formed on two sides of said membrane, wherein said membrane comprises the polymer blend defined in claim 5.

16. The actuator claim 15, wherein said resin is polyvinylidene fluoride.

17. The actuator of claim 15, wherein said metal electrodes are platinum.

18. The actuator of claim 15 wherein said ionic electroactive graft copolymer has a number average molecular weight of 80,000-350,000, or a weight average molecular weight of 144,000-700,000.

19. The actuator of claim 15, wherein n=0 and m=2.

20. The actuator of claim 15, wherein Q is —SO₃⁻.
